(19) United States
(12) Patent Application Publication
(10) Pub. No.: US 2016/0328530 A1
(43) Pub. Date: Nov. 10, 2016

(54) METHOD AND APPARATUS FOR AUTOMATED PATIENT SEVERITY RANKING IN MASS CASUALTY INCIDENTS

(71) Applicant: Umm-Al-Qura University, Makkah (SA)

(72) Inventors: Emad Felemban, MAKKAH (SA); ADIL A. SHEIKH, MAKKAH (SA); Hattan Bojan, MAKKAH (SA); Abdelmajid Khelil, MAKKAH (SA)

(21) Appl. No.: 14/707,423

(22) Filed: May 8, 2015

(51) Int. Cl. G06F 19/00; A61B 7/00; A61B 5/0205; A61B 5/01; G08B 27/00; A61B 5/00; A61B 5/145; H04W 4/00; G08B 21/10; A61B 5/0402; A61B 5/11

(52) U.S. Cl. CPC G06F 19/3418; A61B 5/0402; A61B 7/00; A61B 5/0205; A61B 5/01; A61B 5/11; A61B 5/7271; A61B 5/14532; A61B 5/0006; A61B 5/0008; H04W 4/005; G08B 21/10; G08B 27/001

(57) ABSTRACT

A single disaster could leave hundred thousands of people injured or even dead. In order for the rescue team to reach this knowledge they should send one of the team members to investigate the scene. Body Sensor Networks are emerging systems that can be easily used to measure patient's physiological data and communicate relevant data to other patient using their smart phones. We propose to use Bloom filters, a space efficient probabilistic data structure, for efficiently collecting the dynamic status of patients in a mass casualty scenario. The collected data is disseminated to all nodes in the network to make it available to the rescue team wherever they arrive. In particular, we show that the members of the most urgent cases found at each node are found to be very close to the set of the actual urgent cases.

### FIELD OF TECHNOLOGY

The present invention relates to a method that exploits the Patient to Patient communication to identify the patients' urgency levels and communicate proactively to the rescue team when a Mass Casualty Incident (MCI) manifests.

### BACKGROUND

A mass casualty incident (MCI) is any incident that leaves many people ill or injured, incidents either caused by natural disasters such as floods, tornados and earthquakes or caused by accidental disasters such as explosions, chemical Spills and Plane crashes. The term MCI is specifically used to describe incidents in which emergency medical services (EMS) resources, such as personnel and equipment, are overwhelmed by the number and severity of casualties [1.]

The related massive numbers of deaths have highlighted the need to improve the MCI management. Research and methods from inventors have targeted towards developing emergency management plans for MCIs. However, most of the results were concerned with the role of the rescue team themselves on how they are trained and how to prepare hospitals for large number of patients or to distribute the patients among several hospitals. Disclosed here in is a method to clearly bring the information proactively to rescue team about the MCI victims. The patent application focusses on using the advances in information and communication technologies to make the task of the EMS easier. Many of the casualties died either because the rescue team was unable to locate them given their large number, or the paramedics may have directed their attention to less severe cases since they were not aware of the patient's urgency levels.

### SUMMARY

The aim of our disclosure is to provide communication between patients and the EMS and provide them proactively with the information needed to rescue the patients. All the embodiments mentioned below and in the detailed description use wireless patient to patient communication to transfer important health information from Body sensors to hand held devices. In one embodiment, we use the patient's smart phone as a means of communication. Medical Body Sensor Networks (BSN) is used as an indicator for each patient's condition [1.]BSNs are worn by patients to measure their ECG, PCG, EDG, pressure, pulse monitor, temperature, and motion detection. A BSN connects to the smart phone of the corresponding casualty which enables Patient-to-Patient (Pa2Pa) communication through a Mobile Ad hoc Network (MANET).

In all embodiments, the disclosure takes advantage of Pa2Pa communication to support solving the problem of how the paramedics would know which patients are most urgent, in what order would they start curing the patients. The core objective of our invention is how to identify patient's urgency levels in mass causality incidents. In one embodiment, through communication in the MANET, BSNs can share the medical information of the patients. If we assume that BSNs possess a semantic knowledge on how to use the patients information and output a tagging class according to his/her urgency level such as using [2], then we would have the ability to provide the paramedics with the ranking information. Thus, instead of waiting for the first arriving unit at the scene of the incident, to declare it as an MCI, and examining all the cases to determine which are the most urgent, the rank would be ready as soon as they arrive. In another embodiment, we group the casualties into a few classes; we do not target a full ranking of patients, but a membership capture of each patient to a predetermined criticality class [2]. In another embodiment, we perform safety-critical membership capture process with highest accuracy given that: (i) the data provided is distributed data, i.e., there is no central entity to collect and compare data provided by each patient, (ii) the data is highly dynamic as patient severity could change in seconds, and (iii) there is a tremendous large number of patients which makes it even harder to communicate.

The main disclosure provides the methodology to solve the ranking problem while fulfilling communications requirements. In one embodiment, we concentrate on reducing the communication overhead optimizing both power and bandwidth, since dealing with MANET imposes some limitations including limited bandwidth, unstable network topology, expensive routing and constrained resources, when searching for a suitable data aggregation technique. In another embodiment, our invention is applicable to a very large network with thousands of nodes and enormous amount of data. Since statistical analysis and data mining techniques require heavy processing and powerful data stores, and since we are only interested in computing a single metric that is the class membership we considered probabilistic data structures that can calculate a sufficient estimate of this metric. This estimation serves to reduce the memory overhead at the cost of precision.

In one embodiment, data management is optimized before collecting the membership data as the main aim is to optimize both memory and bandwidth overhead since we are dealing with a tremendous amount of data that have to be sent over a bandwidth-power limited network, namely MANET. Probabilistic data structures for calculating an estimate of the membership is used as they tend to reduce the space consumed at the cost of precision of the final estimate. There are different big data structures [3] that could be used to develop a solution for our problem: Linear Counter (Cardinality Estimation), LogLog Counters (Cardinality Estimation) [4], Hyper LogLog Counters [5]. According to [3] LogLog counters have the least space requirements as they aim at finding the distinct number of elements in a set with repetitions. It can traverse the whole set (or the graph as suggested by Paolo [6]) multiple times and then eliminate the repetitions and out put the count of distinct element. However, this approach cannot be tolerable in our network since there aren't enough resources, bandwidth or power, to allow traversing the network a number of multiple of times. In addition counting is a subset of membership information, and we would still need another structure to store the corresponding IDs. In this disclosure, Bloom filters that allow for a full range of membership functions and have a reasonable memory size without a big loss of accuracy is used. In particular, Bloom filters support membership storage and querying. In this invention, we propose to use the Bloom filter (Bf) structure.

In this invention, an MCI where a large number N of casualties/Patients need to be treated by a number of responders R who require 10′s of minutes to hours to reach the MCI area is considered. Usually N significantly out counts R, i.e., R≧≧N. In one embodiment, we assume that patients are static, nomadic and mobile and that each patient is equipped with the same BSN and wearable sink such as a smart phone. As the incident destroyed the communication infrastructure, only local short range communication is possible between the patients.

In one aspect, we assume that each BSN is identified by a unique ID and its location and is able to communicate with the other surrounding BSNs through a short range communication technology such as WiFi-Direct, BLE and zigBee. In such a MANET, we refer to each BSN/sink pair as a node. We consider that every node knows the ID and location of all other nodes. This knowledge can be easily acquired for instance as follows: Once all the BSNs are connected, each node could broadcast its ID and location to all the nodes in the network. BSNs generate and transmit trustworthy data. Messages may get lost, however, node/link failures and network partitioning are not considered.

Patients are tagged according to their criticality into a few set of classes. We assume that a smart phone is able to compute the criticality class of the corresponding patient from the BSN sensor readings, e.g., using one of the approaches presented in [2]. In one embodiment, the criticality of patients may change quickly, as patient condition might degrade at any instant. To collect the required data from the network, one can use an efficient gossip-based technique that floods throughout the network collecting patients of the most urgent class in the Bloom filter. Finally, copies of the Bloom filter containing the nodes with the most urgent cases will be available on all the nodes in the network.

This invention develops a membership data structure that allows capturing and querying the membership of a certain patient to a certain criticality class in a tremendously large set of data. In another embodiment, some non-safety-critical in accuracies are tolerated. The application data is distributed in the MANET so that the capture should be in a distributed manner.

Bloom Filter Basics: A Bf is a space-efficient randomized data structure for representing a set. It is used to test whether an element is a member of a given set or not. A Bf is an array of m bits (initially set to zeros) used to represent a set of n elements. A Bloom filter uses k hash functions with a range from 1 to m. The equation below shows the relationship between n, m and k

$$k = ( \frac{m}{n} ) * ln 2 \qquad Eq 1$$

In order to insert an element in the filter, apply k hash functions to the element and set the corresponding indices of the filter to 1. To query for an element apply the k hash function and test whether the position corresponding to the resulting indices were set to 1. If one of these positions has a zero then the element doesn't belong to the set. If all of the positions are is then the element may be in the set. This uncertainty results from collisions, multiple elements can map to the same index after applying the hash function to them. Thus a 1 in a certain position doesn't necessarily belong to the element we are querying. This is considered as a false positive. Probability of false positives can be controlled at the cost of increasing the space. False positive probability is inversely proportional with m and directly proportional with n and k and is calculated given Equation (2).

$${( 1 - {e}^{\frac{k ( n + 0.5 )}{m - 1}} )}^{k} \qquad Eq 2$$

There are many variations of Bf. Counting Bf [7] is used if one wants to insert or delete elements form the filter, since each entry in the Bloom filter is not a bit but a counter that is incremented whenever the value is hashed to a certain index. If two values are hashed into the same index, deleting one of them by simply decrementing the count wouldn't affect the existence of the other. Compressed Bf is another type that yields the same false positive probability with smaller number of bits. Scalable Bf adjusts its size to the number of elements stored.

Patient to Patient Communication: The idea of Patient to Patient communication (Pa2Pa) was proposed in [1]. In one embodiment, this technique is used to enhance the current emergency response processes through taking advantage of the BSNs to provide an updated diagnosis for patients' conditions through building Cloudlets that are able to connect together forming a MANET. Patients are ranked according to urgency levels in an automated way by replacing the human knowledge through ontologies. The ranking tasks are divided into smaller tasks starting with collecting live sensor data, then ranking the patients and then scheduling their treatment order for the rescue team present at the scene. The highlights of [1] are: (i) Data quality: the system should be able to adapt to different levels of accuracy of the given sensor data, (ii) Massive data: high data rates generated by the sensors, (iii) Extremely high and varying density of nodes: the number of patients is too large in MCIs and this requires an efficient data transmission strategies given the limited bandwidth. Moreover, patients might join or leave the network causing a varying density of the network. In our disclosure, we would propose a methodology to collect this rank through patient to patient communication, with overcoming most of the major challenges proposed.

Ranking Methods in MANETs: In [9], the authors propose a decentralized ranking algorithm for P2P networks, which uses neighborhood maps to approximate the rankings calculated by link analysis. A node forms its neighborhood map to measure of how good the past behavior of a node depending on how close a neighbor is, and then ranks the nodes accordingly. The algorithm also resolves the security issue that one of the nodes creates attacks that aim to maliciously boost or decrease the reputation of some nodes. Although this algorithm calculate the rank in a distributed way, it is designed for small P2P networks since the storage space needed for each node is n where n is the network size and in our case the n is too large. In our invention, we have a large network size to cater to MCI. In addition, having an unstable topology like the one we are dealing with would mean that each node has to continuously form its neighborhood map and this consumes a large amount of energy for a node in the MANET to handle.

Using Bloom Filters in Ranking: Bf has been proposed for many distributed applications such as web cache sharing [10] and routing [11]. One of the most related works is the ranking algorithm suggested by [12] in which it proposes a gossip-based reputation system (GossipTrust) for fast aggregation of global reputation scores. [12] Leverages a Bf based scheme for efficient score ranking. However, none of these applications has considered safety critical applications with highly dynamic set membership in less dependable and limited resource networks such as patient ranking using MANETs in MCIs.

Overview of the Solution and Methodology: In our disclosure, given a MANET network formed between Patients at the scene of the MCI, our invention will output a rank of the patients with the most urgent on top. In one embodiment, the rank should be reached in a matter of a few minutes (it should be ready upon the arrival of the paramedics) which means that the accuracy and precision required is maximized. In one embodiment, the rank is collected using probabilistic data structures.

In one embodiment, the BSNs are left to communicate to extract the most urgent patients from the network. The invention uses a query based process, upon the arrival of the paramedics a query is sent to a node in the network and this node returns the Bloom filter with the most urgent cases. The communication deals with variable data streams as patient's conditions vary in a matter of seconds since it is an MCI with the possibility of casualty reaching thousands. In one embodiment, the system alerts with updates and reacts quickly. In another embodiment, all the nodes are notified with the updates, to be ready to reply to the rescue teams' query with the most updated version of the rank.

In one embodiment, the patients' conditions would become more critical but not less. As a result, our disclosure only supports join requests to the most critical class but not leave requests from it. In another embodiment, if a patient sends a leave request, the ID would still be stored in the Bloom filter and treated as a false positive. Counting Bloom filters are used to support leave requests, since regular Bloom filters don't support deletions. However, Counting Bloom filters require a larger size as each position represents the counter instead of a single bit. In one embodiment, ID is deleted from the filter and condition is moved out of urgent class, when a patient condition stabilizes.

In one embodiment, the process of attaining the rank can be decomposed into three phases: rank storing, nodes communication and rank querying, which we detail in the following.

Rank Storing: In order to attain the rank using Bloom filter the term rank is simplified to a membership to a class of urgency. In one embodiment, each class represented by a Bloom filter holds all the patients from that class, since BSNs classify patients into urgency classes from the readings of the patient's physiological data. Membership queries can then be performed on the Bloom filter to find out whether a certain patient belongs to a specific class. Rescue team focusses their attention to the most urgent class since the number of patients is enormous which means that the number of patients in the most urgent class will be large as well. In another embodiment, paramedics send a request asking for the Bloom filter which contains the most urgent cases so that they can rescue them first. In one embodiment, paramedics request Bloom filters of less urgent classes if needed. The use of Bloom filters definitely reduce traffic, instead of sending the IDs of all patients that would require a lot of space Patients IDs are hashed into a Bloom filter. Each ID is represented by k indices which are much smaller than the size of the ID which will save a lot of space and thus minimizing traffic as this Bloom filter would have to be sent to all the nodes in the network. Moreover, Bloom filters have no false negatives meaning that there is a zero probability of an urgent patient being neglected; when a membership query returns not found for a patient this patient is certainly not urgent. However, Bloom filters introduce false positives, which will result in dealing with a non-urgent case as an urgent one, but their probability can be controlled by changing the size of the filter.

Nodes Communication: In one embodiment, nodes communicate with each other in a distributed manner to reach the rank. This communication is constrained by the limited bandwidth, power and the communication range of each node as we are dealing with a MANET. Nodes can directly reach only nodes in its communication range. While searching for a suitable communication technique, nodes collect the rank along the way. Aggregation techniques are used to determine the final ranking value, since metrics are collected node by node. In one embodiment, Gossip based aggregation and the tree based aggregation is used as aggregation techniques.

Tree-based Aggregation: It is a tree based routing scheme one node is chosen to be the root , the root initiates the aggregation by forming a Bloom filter and then forwarding a message that contains the Bloom filter and its ID down the tree. In one embodiment, each node that receives the message for the first time sets the transmitter ID as its parent ID keeps forwarding the message down the network. The message keeps flooding down the tree until all the nodes are assigned a parent. Each node would specify a listening interval during which it listens to queries from its children. If a child is an urgent case the child hashes his ID into the Bloom filter and sends it to his parent. The parent then unions the Bloom filters received from its children and store the result in its own Bloom filter. Aggregates would keep flowing up until they reach the root which will have the Bloom filter with all the urgent cases. The root node broadcasts the final aggregate to all nodes since all the nodes will have a copy of the final aggregate.

Gossip-based Aggregation: The aggregate is collected through broadcasts; a node initiates the broadcast by sending the broadcast message that contains a Bloom filter for the urgent cases to its neighbors. In one embodiment, neighbors communicate urgent cases to neighbors till the whole network is traversed using communication protocol such as MANET. If a node belongs to the most urgent class the node hashes its ID to the Bloom filter and continues the broadcast with the updated Bloom filter. Nodes who last receive the broadcast message with the most updated Bloom filter broadcast back their copy of their Bloom filter to all the nodes in the network. In another embodiment, this phase has communication costs high.

After considering both approaches, in one embodiment, gossip based approach is chosen as it is more robust to node failures, mobility and frequent changes in the sensor data though it usually consumes more node energy and bandwidth. In another embodiment, broadcasting is further optimized by using probabilistic gossiping since the nodes are densely distributed meaning that the nodes are so close by, so one broadcast might cover a large percentage of nodes and there only a small fraction of nodes still need to forward the broadcast message [13].

Rank Querying: The rank is queried by the rescue team as soon as they reach the scene of the MCI. The rank can be requested from any node at the scene. The rescue team queries the rank from the first patient they attend to, the patients BSN smart phone connection returns the Bloom filter with the most urgent cases. In one embodiment, the rescuers have a full list of IDs of all patients to test the patient's membership to the Bf and find the most urgent patients and their locations. In another embodiment, the smart phones of most urgent patients display a sound so that static patients can be physically easier identified.

Critical Cases Membership Collection: In one embodiment, the BSN worn by the affected member in MCI relays the critical vital body statistics to the hand held device. The hand held device uses MANET protocol to communicate with other devices around its broadcast radius. In one embodiment, a random node S initiates the aggregation. Node S generates the Bloom filter with all the bits initially set to zero, the Bf will hold the IDs of most urgent nodes. Node S then broadcasts the Bloom filter to its neighbors and neighbors to their neighbors until the whole network is traversed. Only nodes with the most urgent cases report their IDs into the Bloom filter. In one embodiment, when the initial broadcast is over, the nodes at the edge of the network, i.e., the nodes that last received the Bloom filter will now have an approximate of the global aggregate of the urgent cases.

Criteria for choosing edge nodes: A node is considered an edge node if it receives repeated acknowledgment (ack) from all its neighbors. In one embodiment, a node receiving a Bloom filter indicates that the broadcast has reached it. A node sends a repeated ack when it receives another Bloom filter, whether it's the same as its current Bloom filter or different, it sends the repeated ack to the source of the broadcast. If the source of the broadcast receives repeated acks equal to the number of its neighbors, then the source determines that further broadcast is not needed as all the nodes were already reached. Consequently, this source is chosen to be an edge node. In another embodiment, assume we have L edge nodes in order to update the rest of the nodes with the collected data; these edge nodes broadcast their copy of their Bf with a certain flag to the rest of the nodes. To get better approximate of the global aggregate, nodes that receive L different approximates of the global aggregate from the edge nodes gets the union of these different versions by ORing [4] all the bits together to acquire the global aggregate for the whole network (Note that the node only unions the received Bloom filter if it was flagged as sent from an edge node).

In one embodiment, the method keeps track of patients changing their urgency class especially that the patient condition could degrade in any second. However, we are only concerned with the nodes that reached the most urgent class. The node which has changed its class would raise a flag. Accompanied with raising the flag a node should first hash its ID to its Bf then sends a broadcast update message to all the nodes in the network. Update messages can only be exchanged after the broadcasting phase is over and all the nodes have a copy of the global list of the most urgent nodes.

Node failures affect the performance of the method since it is based on broadcasting, and it slightly affects the global aggregate if it was one of the most urgent nodes (the accuracy of the aggregation is directly proportional to the number of losses). In another embodiment, if a lost node tries to reconnect with the network, it sends a rank query to one of its neighbors requesting the so far collected rank (its current copy of the Bf) and proceeds like any other node. If this node was is in the most urgent class after receiving the copy of the Bf and after assuring that the broadcast phase is over, it hashes its ID to the Bf and sends an update message to the rest of the nodes.

Other features will be apparent from the accompanying Figures and from the detailed description that follows.

### BRIEF DESCRIPTION OF THE DRAWINGS

Example embodiments are illustrated by way of example and no limitation in the graph and in the accompanying Figures, like references indicate similar elements and in which:

FIG. 1 illustrates the gradation in casualty incidents.

FIG. 2 shows the how various technology help in speeding up patient information to be available proactively when the rescue team arrives.

FIG. 3 provides a prior art on the acute casualty arrival time distribution after a Mass Casualty Incident (MCI).

FIG. 4 shows how the MCI resolution time impacts the MCI casualty.

FIG. 5 shows the medical Body Sensor Networks (BSN) capturing important patient information through sensors embedded in the patient body and relaying real-time to the wireless unit.

FIG. 6 illustrates the Patient to Patient (Pa2Pa) communication through Mobile Ad Hoc Network (MANET).

FIG. 7 shows the Body Sensor Network's data broadcast sphere of influence.

FIG. 8 shows the proposed broadcast aggregation to reach rescue team using MANET.

FIG. 9 shows the proposed methodology to determine by the rescue team upon arrival, the most urgent patients.

FIG. 10 shows the methodology to collect urgent cases through broadcast in MANET.

FIG. 11 illustrates the false positive counts among the distribution of nodes.

FIG. 12 illustrates the false negative counts among the distribution of nodes.

FIG. 13 illustrates the impact on message loss rate on communication overhead.

FIG. 14 illustrates the impact on message loss rate on latency.

FIG. 15 illustrates the impact of message loss rate on false positives and false negatives probability.

FIG. 16 illustrates the impact of accuracy in terms of number of nodes over a number of collected urgent cases.

FIG. 17 illustrates the impact of message loss rate on communication overhead with updates over a larger sample set.

FIG. 18 illustrates the impact of message loss rate on latency over a larger sample set.

FIG. 19 illustrates the probability of loss verses false positives-false negatives over a large sample set.

Other features of the present embodiments will be apparent from the accompanying detailed description that follows.

### DETAILED DESCRIPTION

The present disclosure relates to a method and system to accurately relay proactively affected patient's vital body information taken through Body Sensor Network (BSN) and relayed to the smart device near the patient, and through MANET to other devices around. The devices also use the submitted ranking methodology to calculate the most urgent cases. A query mechanism is also submitted that makes it easy for the rescue team to pull the urgency data from any of the smart devices available in MCI.

Example embodiments, as described below, may be used as a method, process and system to atomically structure the data query management of the rescue team for attending the most urgent cases first.

It will be appreciated that the various embodiments discussed herein need not necessarily belong to the same group of exemplary embodiments, and may be grouped into various other embodiments not explicitly disclosed herein. In the following description, for the purposes of explanation, numerous specific details are set forth in order to provide a thorough understanding of the various embodiments.

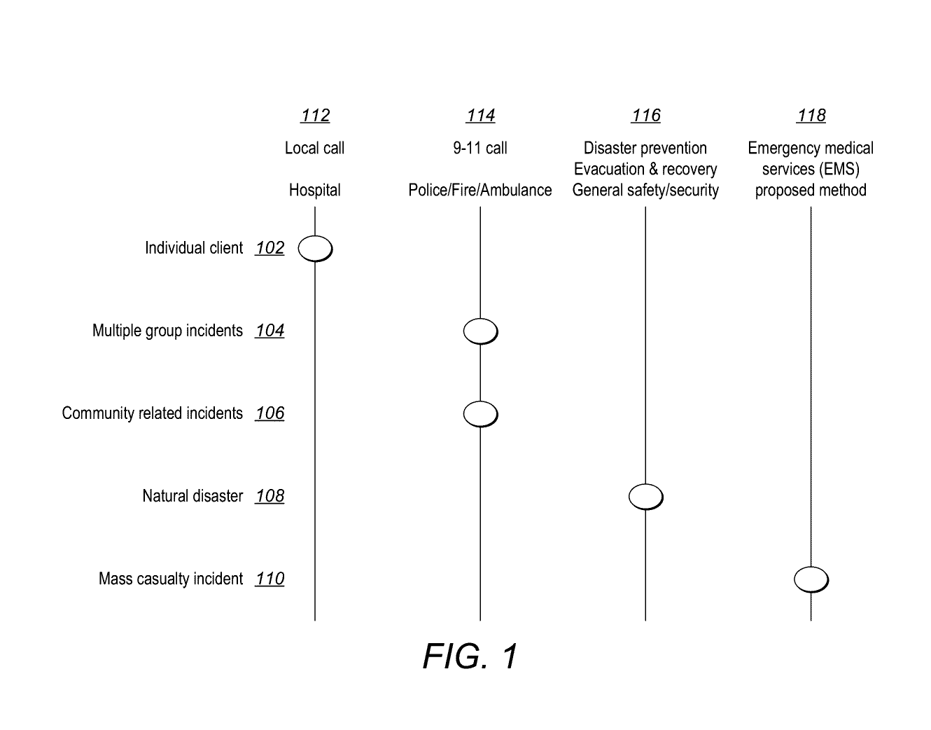

FIG. 1 shows the gradation in casualty incidents. Not all the incidents can be classified as Mass Casualty Incident (MCI). Hence, the action that is taken is commensurate to the safety and security impact that effect has created. For example, an individual incident 102 normally leads to a local call for help to medical center or hospital 112. However, when a multiple group incident 104 happens such as a car accident, a 9-1-1 call to bring Police, Fire and Ambulance 114. Similarly, community related incidents 106 end up being serviced by local police, fire or ambulance 114. On the contrary, when a natural disaster such as flood, hurricane and tornado occurs 108, disaster management kicks in 116. Disaster management includes comprehensive prevention, evacuation and recovers planning for general safety and security of the public. Natural disasters could lead to Mass Casualty some times. Typical Mass Casualty Incident (MCI) 110 happens unexpectedly due to negligence or planning. For example, terrorist attack or bridge collapse comes unannounced. This is where Emergency Medical Services (EMS) 118 kicks in full force. EMS requires the patient data as soon as they arrive, and the proposed method enables quick gathering and ranking of patient data for patient handling prioritization.

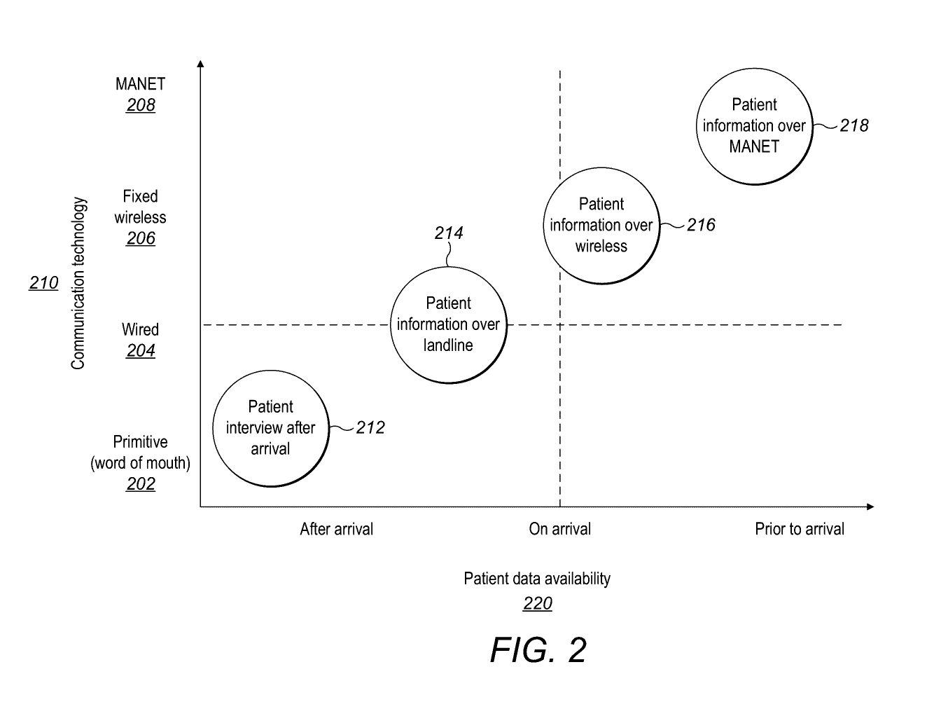

FIG. 2 shows the technology progression 210 with respect to communication of patient data over a period of time 220. In one or more embodiments, the patient information over wireless 216 may be coupled with other mediums such as cellular or satellite. In earlier days 202, when a Mass Casualty Incident (MCI) occurs, the rescue team arrives there without knowing what to expect. The patient information was mostly available after arrival, and the patient conveyed to the rescue team his or her discomfort directly 212. When wired communication such as land line telephones 204 was available, some patient information and the level of impact was conveyed over the land line to the nearest authorities 214, who in turn conveyed to the rescue team. However, the sheer volume of all patient data was not available to the rescue team until they arrived on-site. The prioritization of patients according to criticality was done by the rescue team after collecting information about patient and classifying them. This took a long time. In some places, later fixed wireless 206 was available to convey the information, which was used by the authorities to prepare 216 their plan of action prior to their arrival. However, the number of critical patients and their identification were not available until the rescue team arrival and completion of analysis. The proposed method uses Mobile Ad Hoc Network (MANET) 208 to collect the patient data proactively. Using Body Sensor Network (BSN) and MANET, patient information can be analyzed and ranked according to criticality 218 and made available. The rescue team can read this information and start the work immediately. In another embodiment, MANET may be substituted for another wireless medium such as infrastructure mode (WiFi, 802.11 a/b/g/n/ac) for communicating.

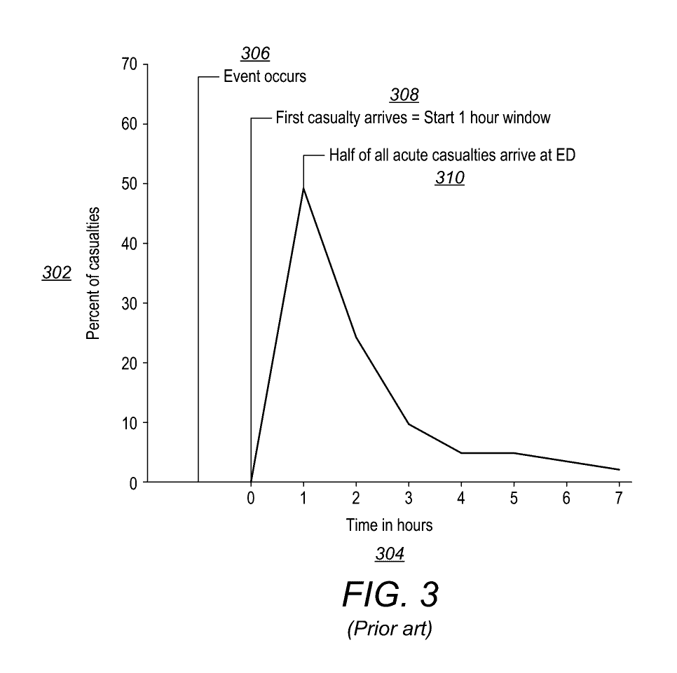

FIG. 3 shows a PRIOR ART of the arrival time distribution after a Mass Casualty Incident (MCI). The data collected by the US Center for Disease Control and Prevention, Emergency Preparedness and Response shows that when an event occurs 306, the first casualty 308 arrives at the start of the hour window. When the percentage of casualties is measured 302 versus the arrival time 304 of casualties, it can be seen almost half 310 of the acute casualties arrived at emergency to be seen by the rescue team. This shows that the importance of handling the affected patients within the first hour to decrease the fatality rate. For this to be done, it is important to segregate the patients that require urgent care, and have their information ready when the rescue team arrives. Maximum amount of damage results when the rescue team arrives and reactively collects data to determine which patient needs urgent care and where he/she is. Better communication and availability of data decreases the response time.

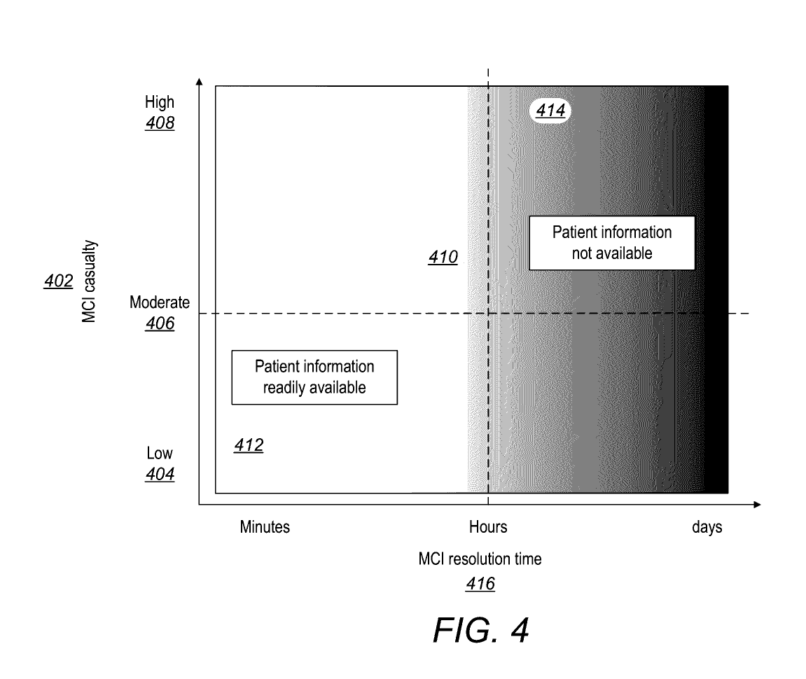

FIG. 4 illustrates the patient information availability and its impact on incident resolution time. When a Mass Casualty 402 occurs, the casualty can be low 404, moderate 406 or high 408. The casualty rate cannot be controlled by the rescue team; however the fatality rate can be controlled to an extent if the patient information is readily available. In general, we see the MCI resolution time 416 follows an S-Curve when a Mass Critical Incident occurs, where the casualty starts to rise slowly during the minutes of first hour 412, and then exponentially increases within the next hour 410 or so before stabilizing over a period of time 414. If the patient information is readily available when the rescue team arrives within the first hour, the fatality rate can be contained. If the information is not readily available, then the rescue team is busy collecting information and grading the seriousness of patients while the fatality is increasing.

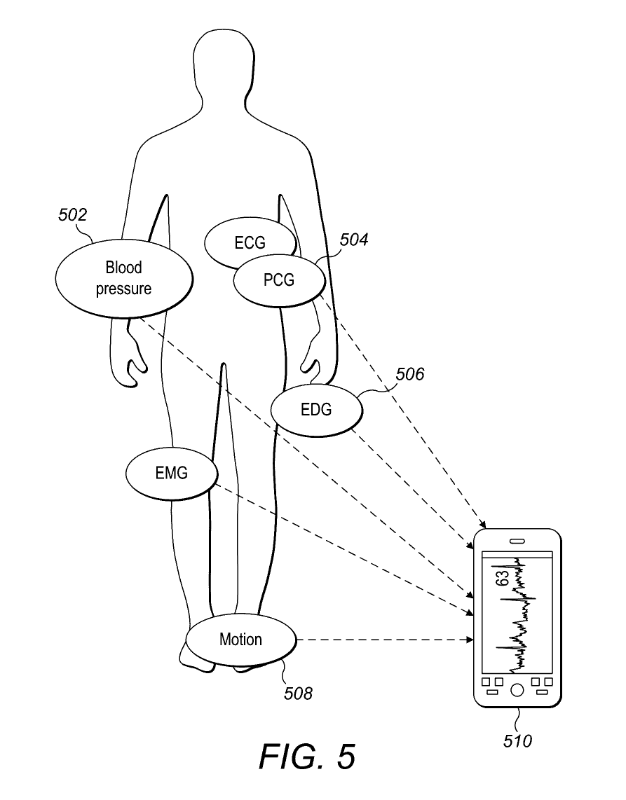

FIG. 5 shows the medical Body Sensor Networks (BSN) to capture patient information. The patient who is a casualty is wearing the sensors; some are shown in Figure, such as Blood Pressure sensor 502, Echo Cardio Graphic sensor (ECG), Phono Cardio Graphic sensor (PCG) 504, Electro Dermo Graphy (EDG) 506, Motion sensors 508 and EMG. These sensors send data over wireless medium that is captured by a simple hand held device 510. In another embodiment, the BSN could be non-sensor based, providing vital statistics to the smart devices using some wired or wireless interface.

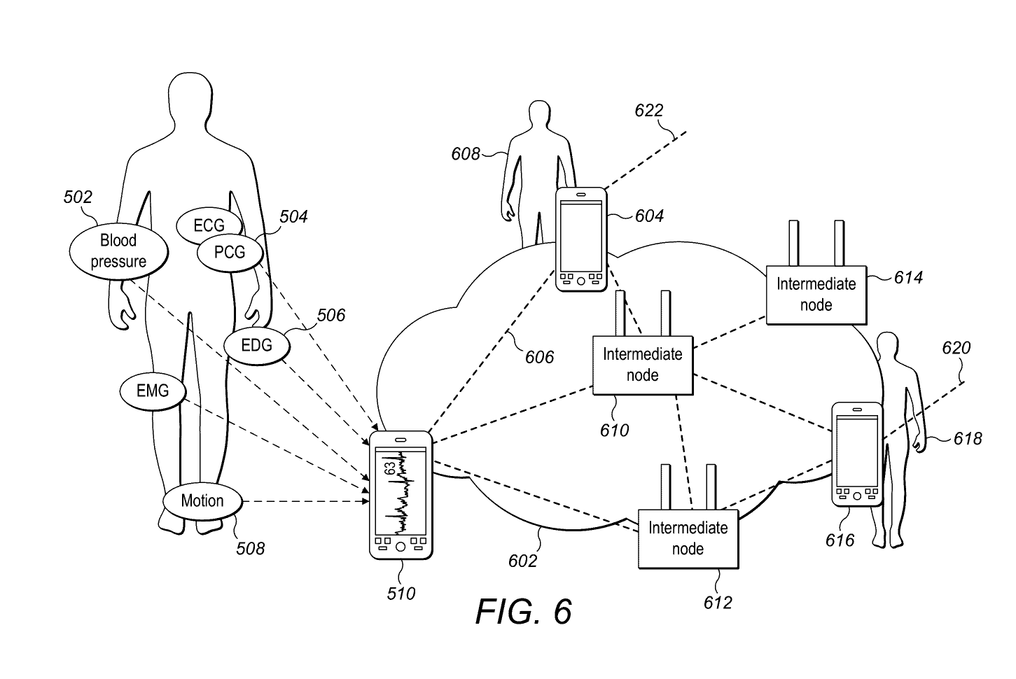

FIG. 6 shows the communication between various devices 510, 616, 604 that are collecting patient information from the Body Sensor Networks (BSN). Every patient is connected to various sensors such as Blood pressure monitors 502, ECG and PCG 504, EDG 506, EMG and Motion sensors 508. The sensors communicate over wireless medium to a handheld device 510 that has the capability of working in an infrastructure mode or an Ad Hoc Mode using MANET protocol. The device automatically connects to other Access Points (also known as wireless nodes or intermediate nodes) through a wireless link 602. A device connected to BSN 510 can directly communicate over MANET to another device 604 connected to another BSN 608. The same device 510 can also communicate with other intermediate nodes 610 also that are not connected to BSN, but can act as a transit nodes supporting MANET. Similarly, the patient information can be communicated between intermediate node 610 and 614. An intermediate node 612 can also communicate over MANET to a handheld device 616 supporting MANET that is connected to a patient 618 over BSN. The MANET carries the patient information including urgency ranking so that the rescue team can pick up the information from any of the nodes quickly when they arrive. The rescue team can connect to any node through a wireless link 620 to pick up the ranking information of the entire affected population that is connected to BSNs. In another embodiment, the network of all smart devices communicates over a well-defined proprietary communication protocol or a wired communication protocol. All the calculations are done using mobile devices, standard processors and regular computers. In another embodiment, not all nodes are connected to a patient, and some nodes belong to those who are not affected. Those nodes, named as Intermediate nodes 610, carry traffic without having a connection to a BSN itself.

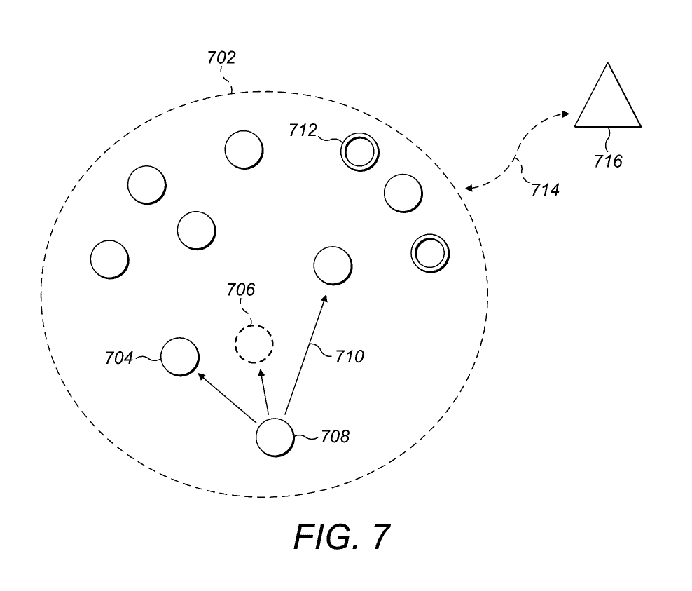

FIG. 7 illustrates the Body Sensor Network (BSN) patient information broadcast sphere of influence. The sensors in the body are transmitting information to the hand held devices, which has a wireless interface to communicate 708 using MANET 710 to the neighboring nodes 704, 706. However due to the limitations of the wireless radio and antenna power, the reach of the signal is limited 702. The broadcast reach is in the range of feet and meters rather than kilometer range. Due to the Mass Casualty Incident (MCI) scenario, the affected parties are close to each other and hence the patient information can be transformed to another node nearby quite quickly using MANET. The nodes 706 can either be static nodes 704 where a patient is stationary or a mobile node 706 where the patient is moving around. The patient information, regardless of the seriousness is communicated to neighboring nodes, so that when the rescue team 716 arrives, they can pull the data through MANET 714 from any node within the sphere of influence. Based on the data, they have the ranking for cases that are urgent 712 and non-urgent 704. In one embodiment, the broadcast radius could be smaller and the devices communicate point to point through a fixed topology. In another embodiment, a node is connected to a router which has connectivity to either an Access Point (AP) or a Customer Premises Equipment (CPE) so that long distance transfer of data to a nearby hospital can be accomplished, without waiting for a rescue team to arrive. In another embodiment, the communication could be from an AP or CPE to a mobile ambulance that is arriving to MCI area.

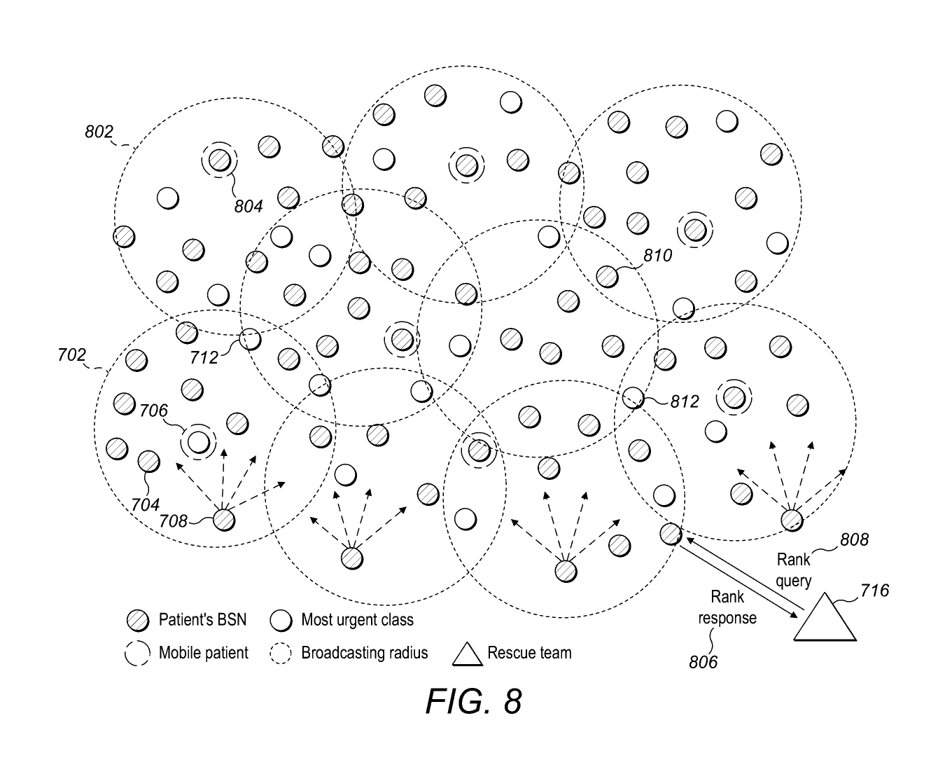

FIG. 8 illustrates the proposed broadcast aggregation to reach the rescue team. Every single wireless node 708 has a sphere of influence 702 based on its radio and antenna power. Some nodes are connected to a patient's BSN 704 out of which some of the nodes are patients that are moving 706 and some are deemed urgent 712. Some nodes 712 can overlap between two broadcasting radius 712, where they can receive and transfer information to nodes in multiple spheres. For example, a patient that is classified as urgent 712, 812 can send or receive in multiple spheres 702, 802 and reach nodes in both spheres 704, 804, 810, 812. The proposed methodology uses the MANET to communicate important patient information from an individual BSN seamlessly through other MANET compatible devices including those of other patients resulting in all wireless devices having information of all the patients. The proposed methodology also incorporates the ranking system where the patients are classified according to their urgency 712, 812. The proposed methodology also provides a mechanism where a rescue team 716, on its arrival can connect to any node within the broadcast radius and rank query 808 to pull the urgency rank data of all the patients, where the any node can rank response back the complete data 806. In one embodiment, the data is sent using a set routing protocol such as Open Link State Routing (OLSR) instead of broadcast, to minimize intra node communication overhead.

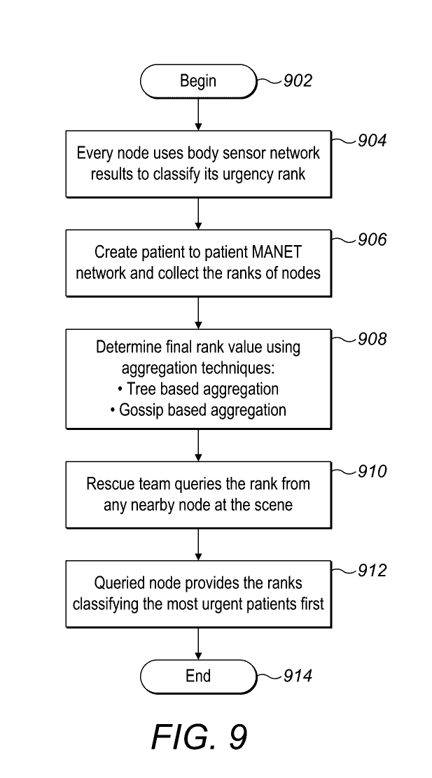

FIG. 9 provides the proposed methodology on the determination of most urgent patients by the rescue team on arrival 902. Body Sensor Network (BSN) is incorporated in every patient through various sensors to measure vital signs including blood pressure, ECG and motion. Every node connected to the BSN takes the sensor results and classifies the urgency of the patient and ranks the patient in terms of urgency 904. Once the information from the BSN is categorized by the wireless node, communication using MANET is established to neighboring nodes to collect the ranks of the neighboring nodes 906. Gossip based aggregation technique 908 is used to determine the final rank value. The aggregate is collected through broadcasts; a node initiates the broadcast by sending the broadcast message that contains a Bloom filter for the urgent cases to its neighbors. Then from neighbors to neighbors till the whole network is traversed. If a node belongs to the most urgent class the node hashes its ID to the Bloom filter and continues the broadcast with the updated Bloom filter. Nodes who last receive the broadcast message with the most updated Bloom filter would have to broadcast back their copy of their Bloom filter to all the nodes in the network. This phase might make the communication costs high. In another embodiment, tree based aggregation method can be used for aggregation. When the rescue team arrives 910, it queries the rank from any of the nearby node at the scene to have the list of urgent patients it needs to attend to. The queried node 912 provides the classification data to the rescue team for them to start the job 914.

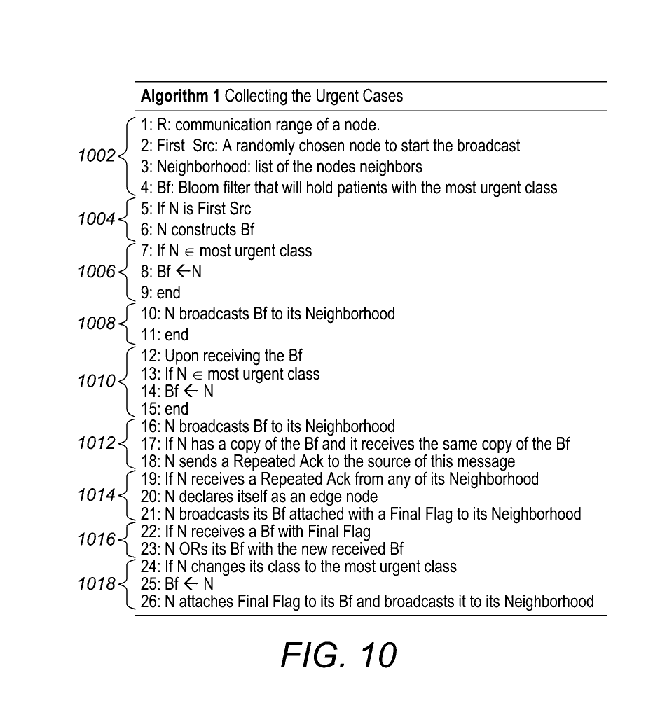

FIG. 10 provides details of the proposed methodology. One random node S will initiate the aggregation 1002. Node S would generate the Bloom filter 1004 with all the bits initially set to zero; the Bf will hold the IDs most urgent nodes. Node S then broadcasts the Bloom filter to its neighbors and neighbors to their neighbors until the whole network is traversed. Only nodes with the most urgent cases 1006 Patient's BSN Most urgent class Mobile patient Broadcasting radius Rescue team should hash their IDs into the Bloom filter. When the initial broadcast is over 1008, the nodes at the edge of the network, i.e., the nodes that last received the Bloom filter will now have an approximate of the global aggregate of the urgent cases. Criteria for choosing edge nodes: A node is considered an edge node if it receives repeated ack from all its neighbors. Receiving a Bloom filter by the node 1010 indicates the broadcast has reached it. A node sends a repeated ack when it receives another Bloom filter, whether it's the same as its current Bloom filter or different, it sends the repeated ack 1012 to the source of the broadcast. If the source of the broadcast receives repeated acks equal to the number of its neighbors, then this broadcast round was useless since all the nodes were already reached and there is no need for further broadcasts. Consequently, this source is chosen to be an edge node. Assume we have L edge nodes in order to update the rest of the nodes with the collected data; these edge nodes would broadcast their copy of their Bf with a certain flag to the rest of the nodes 1014. To get an even better approximate of the global aggregate, nodes that receive L different approximates of the global aggregate from the edge nodes gets the union of these different versions by ORing all the bits together to acquire the global aggregate for the whole network 1016 (N.B. the node only unions the received Bloom filter if it was flagged as sent from an edge node). Our problem now is how we would keep track of patients changing their urgency class especially that the patient condition could degrade in any second. However, we are only concerned with the nodes that reached the most urgent class. The node which has changed its class would raise a flag 1018. Accompanied with raising the flag a node should first hash its ID to its Bf then sends a broadcast update message to all the nodes in the network. Update messages can only be exchanged after the broadcasting phase is over and all the nodes have a copy of the global list of the most urgent nodes. Node failures would not affect the performance of the algorithm since the algorithm is based on broadcasting, it would only slightly affect the global aggregate if it was one of the most urgent nodes (the accuracy of the aggregation is directly proportional to the number of losses). If a lost node tries to reconnect with the network, it will send a rank query to one of its neighbors requesting the so far collected rank (its current copy of the Bf) and proceeds like any other node. If this node was is in the most urgent class after receiving the copy of the Bf and after assuring that the broadcast phase is over, it hashes its ID to the Bf and sends an update message to the rest of the nodes.

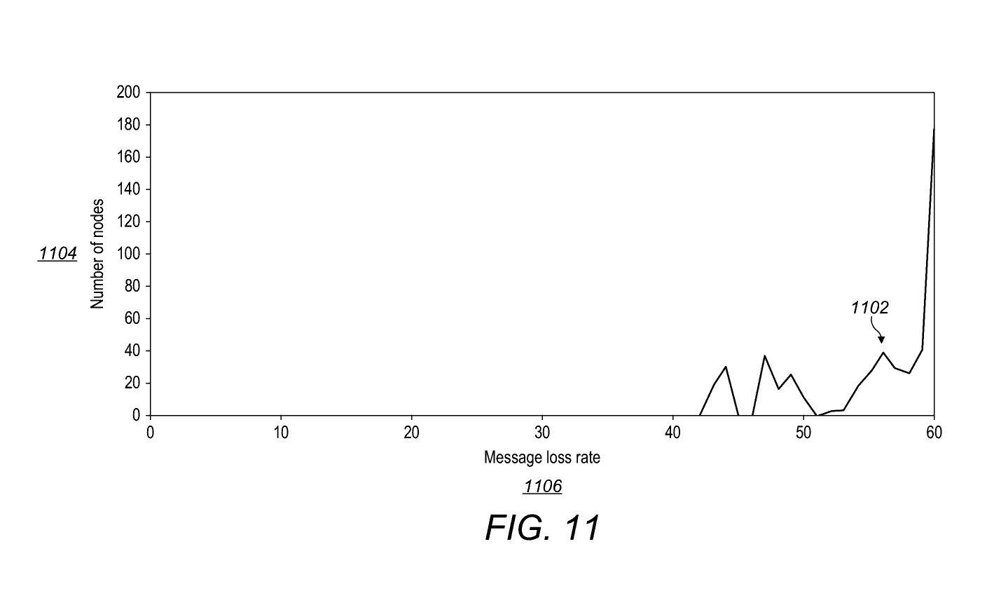

FIG. 11 illustrates the false positive counts towards accuracy and precision: Accuracy quantifies how close is the number of urgent cases collected at each node to the actual number of urgent cases. Given the aggregated number of urgent cases by each node, precision is calculated by measuring how close it is to the number collected by other nodes in the network. FIG. 11 shows the failure free case, the graph shows that most of the nodes 1104 stored the full number of urgent cases in their Bloom filter. The numbers of urgent cases collected by most of the nodes is accurate 1102 since it so close to the actual value of the urgent cases.

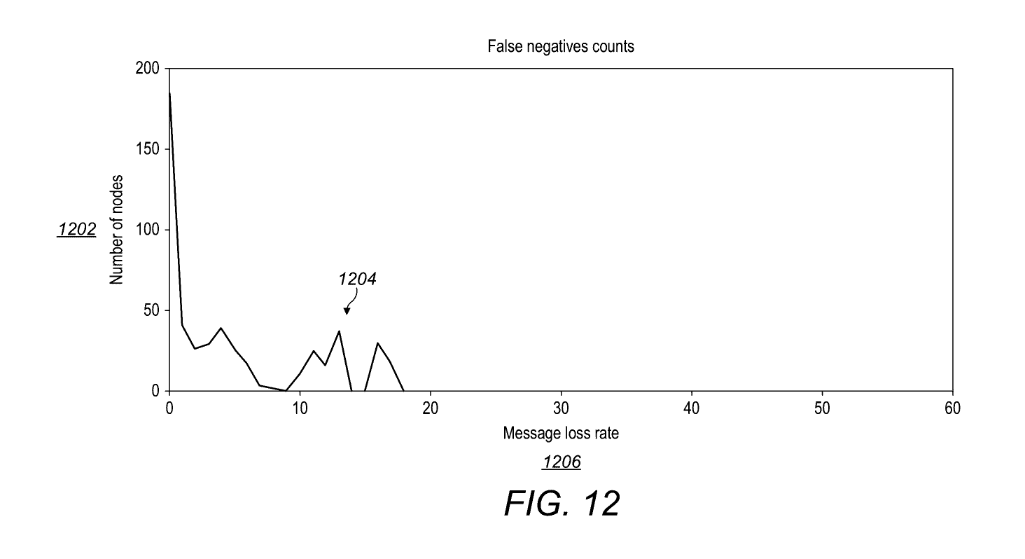

FIG. 12 shows the distribution of the nodes 1202 over the number of urgent cases collected. In addition, our implementation results in a precise count of the urgent cases as the values acquired by the nodes are close to each other as shown in FIG.12. This precision is highly important because it gives the rescue team the flexibility of querying the count from any node and still has a good approximate. The graph shows the failure free case 1204 of the distribution of the nodes according to the false negatives in each of their Bloom filters. False negatives refer to the number of urgent cases missing from the nodes collected data. Since Bloom filters have a zero probability of false negatives, the false positives encountered are definitely aggregation and communication losses. Our methodology was able to achieve zero false negatives for almost 36% of the nodes in the failure free case.

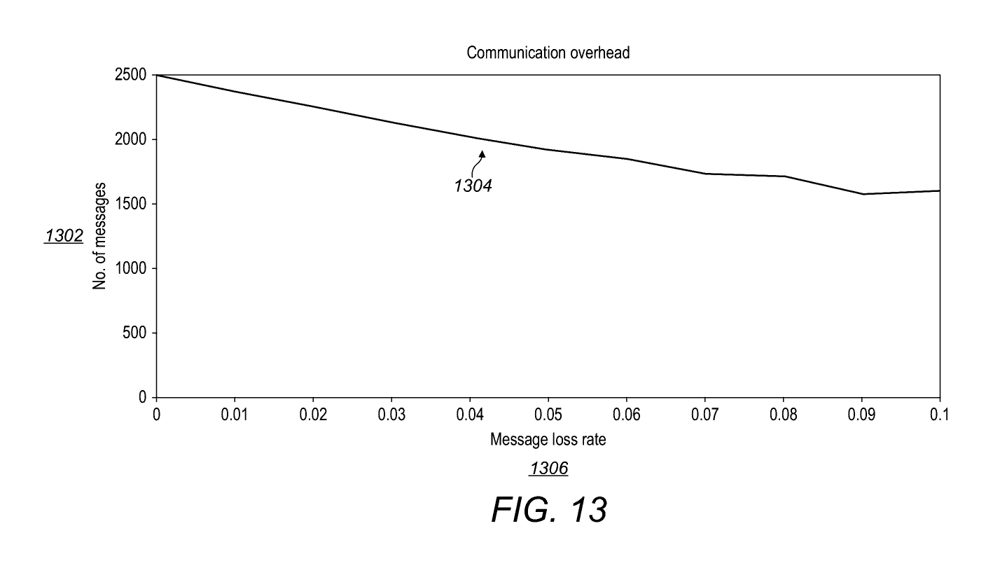

Traffic Overhead and Latency: To evaluate the network traffic we calculated the total number of transmitted messages throughout the whole network with considering message losses. We simulated message losses up to 1% and calculated the number of sent messages 1302 without retransmissions. FIG. 13 demonstrates the decrease in total number of messages 1304 needed to reach the final rank as the percentage of loss 1306 increases.

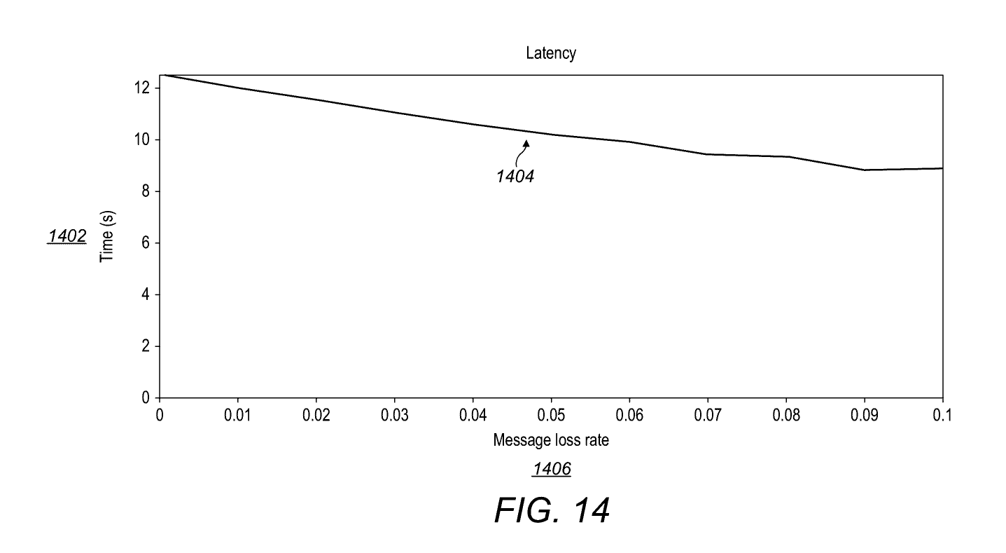

FIG. 14 shows a leap increase in the number of transmitted messages as all the nodes who updated their urgency level need to broadcast their membership. This increase is a bit costly however it assures that no urgent node is neglected and that the update would be available on all the nodes. FIG. 14 also shows the corresponding latencies 1404 assuming that each broadcast message from one node to its neighbors requires 5 ms 1402.

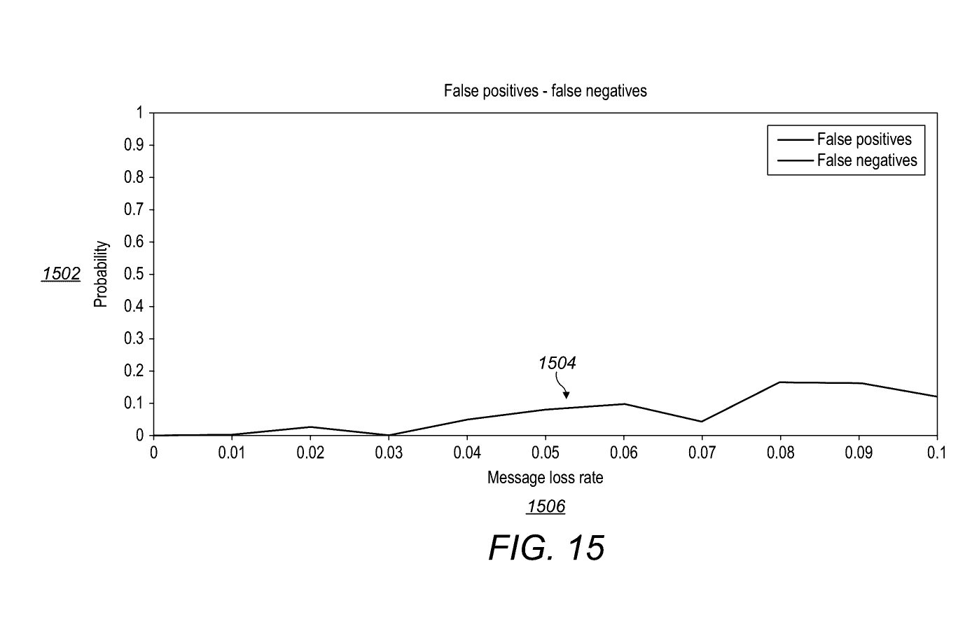

False Positives and False Negatives Probability: The probability of false positives in a Bloom filters varies according to the number of inserted elements into the filter. As the number of inserted elements increase, the number of ones in the Bloom filters increases and consequently the probability of false positives increase. FIG. 15 illustrates the average false positive probability for the network it shows decrease in the average false positive probability as the probability of loss increases 1504. This is expected since the increase in the loss probability results in a failure to deliver the full list of urgent cases 1506, which means that the Bloom filter would hold a fewer number of elements thus the probability of false positives decrease. Along with the false positive probability, FIG. 15 shows the average probability of false negatives which is, as we discussed earlier, a measure of the number of urgent nodes that failed to reach the Bloom filter. Accordingly, the average false negative probability increases with the probability of message loss. FIG. 15 shows the false positives and false negatives probability after the updates. A slight increase in the false positive probability is noticed compared to the graph in FIG. 15. The increase in the false positive probability is due to the increase in the number of urgent cases. However, the false positive probability is the same since it is fully dependent on the message loss rate.

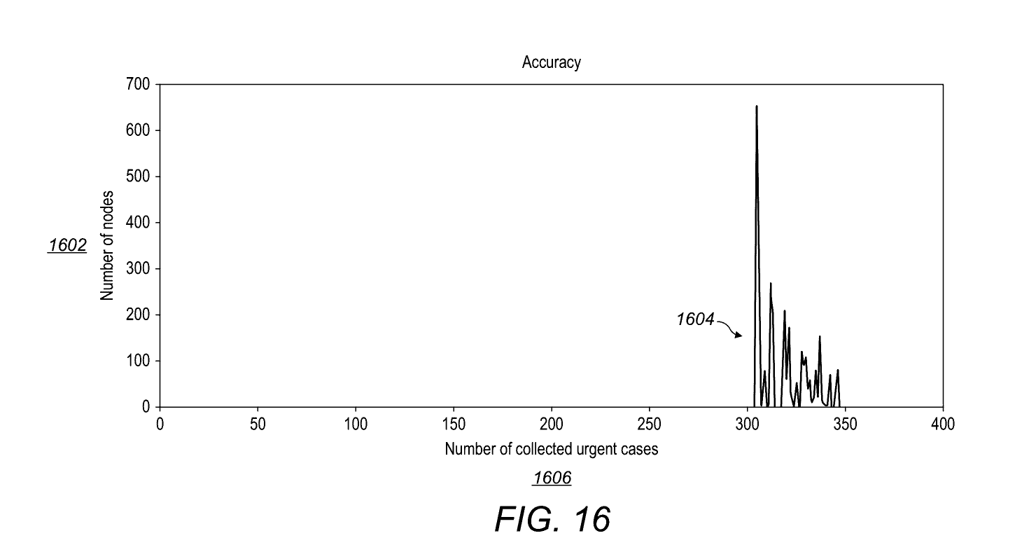

FIG. 16 shows the accuracy measured 1604 during the simulation for a larger number of nodes to reach over 3,000 nodes 1602 using Bloom filter with 0.01 false positive probabilities and a size of 30,297. FIG. 16 shows the accuracy measured for 3000 nodes and it can be noted that the accuracy in terms of collecting urgent cases 1606 do fall due to large network size. Counts by the nodes are roughly within the range from 250 to 300.

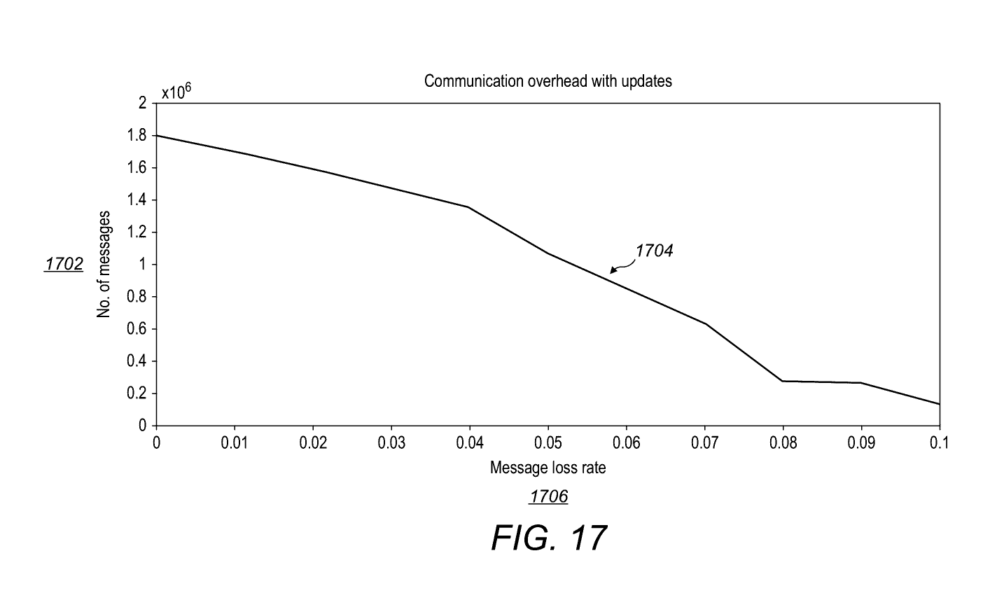

FIG. 17 shows the communication overhead for transmitted messages 1702 when simulated for 3000 nodes. The total number of messages 1704 transmitted through the network when there was no loss was 15000 and 18000 for updates.

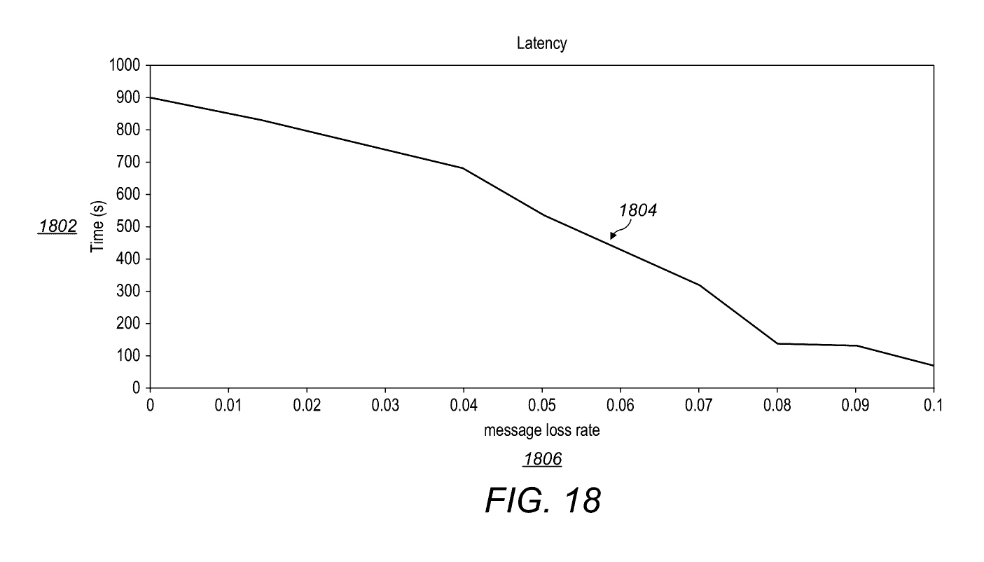

FIG. 18 shows the latency 1802 when simulated for 3000 nodes. The corresponding latencies 1804 were 74.05 seconds for the message loss free case with no updates and 900 seconds for the case of updates as shown.

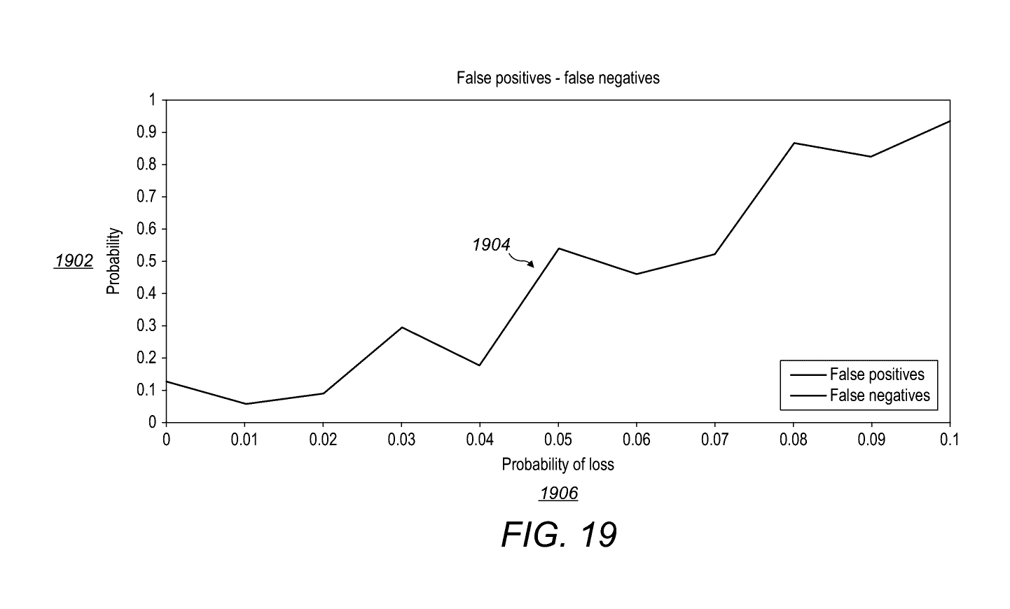

FIG. 19 shows the false positive and false negative probability 1902. The false positive probability is still constrained under 0.01 while the false negative continues to increase 1904 with losses for both the case with no updates and the case with the updates. The increase of the network size yielded the same expected result the only difference was in the slight decrease in the level of accuracy which can be avoided if we loosen the communication constrictions.

Although the present embodiments have been described with reference to specific example embodiments, it will be evident that various modifications and changes may be made to these embodiments without departing from the broader spirit and scope of the various embodiments.

### INDUSTRIAL APPLICABILITY

Accordingly, the specification and drawings are to be regarded in an illustrative rather than a restrictive sense. The invention is valid for all MCI situations and all BSNs that have interfaces to a smart device or any other device that has wireless or wired or any communication devices. The invention works with all smart phones that are equipped with standard applications to interface sensors. The invention does not require any special permission to be generated on the smart devices. The hall mark of the invention is that the innovation works seamlessly and silently in the background without any disturbance to the smart device owners to receive the sensor data from BSN, analyze the data for urgency, relay the data to nearby nodes within its broadcast radius and acknowledge the receipt of the urgency ranking. Please note that the procedure works well with smart devices. The invention is directly applicable to the Emergency Medical Service (EMS), safety, security and disaster recovery markets. In addition, the invention is applicable to homeland and border security, defense and mass entertainment markets. The invention can be applied to the map industry to provide real-time data to hospitals, fire stations, ambulance and police on the MCI events and gravity of situation.

What is claimed is:

1. A method, comprising:
gathering a raw sensor data residing in the body of an affected person;
gathering data in patient to patient network;
gathering data from various sensors in series and/or parallel connected to Body Sensor Network (BSN);
applying intelligence to the data gathered to determine the severity of the affected person; and
calculating the urgency class the person should belong to.

2. The method of claim 1, further comprising:
finding the vital body signs, readings and information such as, but not precluding, blood pressure, heart rhythms, pulse, motion detection, fever, temperature and blood sugar

3. The method of claim 2, comprising:
gathering data from the sensors in BSN and relaying over wired or wireless interface to a smart device or any other networking device that can connect to sensors; and
analyzing data from the sensors from BSN using software that are available over applications or as a separate hardware connected to the smart device or networking device.

4. The method of claim 3, wherein the smart devices or network devices have wireless interfaces that can communicate with other smart devices either in infrastructure mode or in Ad Hoc mode.

5. The method of claim 3, further comprising:
communicating with neighboring nodes using MANET protocol;
communicating with neighboring nodes that are not connected to any sensor network; and
relaying the urgency ranked information to the neighboring nodes.

6. The method of claim 5, further comprising:
communicating with neighboring nodes using a proprietary protocol; and
communicating with neighboring nodes using a routing protocol or broadcast medium.

7. The method of claim 4, further comprising:
determining final rank value using Tree based aggregation mechanism;
determining aggregation using Gossip based aggregation mechanism; and
determining final rank value using rule based methods where intelligence for the rules are input separately based on the geographical and other considerations

8. A system, comprising:
a sensor network connected to or in the vicinity of the body collecting vital body information;
a smart device or a networking device that has a communication capability of working in wired or wireless medium through a general purpose standardized protocol or a proprietary routing protocol;
a methodology to calculate the urgency level;
a methodology to calculate the final ranking of the urgency among the affected members;
a methodology to determine the final correct urgency ranking among the set of nodes;
a query methodology to provide the status of ranking among the group; and
an interface to the rescue team so data is available from any node at any time.

9. The system of claim 5, wherein the connectivity between a subset of nodes can be directly connected over point to point, or point to multipoint wireless network directly to fire, police, ambulance or hospital network to provide up-to-date information.

10. The system of claim 5, further comprising:
a probabilistic ranking method to provide a quick sense of the urgency so the rescue team prepares for the situation.

11. The system of claim 6, wherein the device is an android device, linux device, windows device or an apple iOS device.

12. The system of claim 6, further comprising:
a wireless module in the device comprising of 802.11 a/b/g/n/ac compatible cards;
a wimax module in the device comprising of 802.16 d/e protocol compatible cards;
a HSPA, LTE compatible device; and
a Zigbee, Bluetooth, RFID compatible device.
